(19) United States
(12) Patent Application Publication
(10) Pub. No.: US 2005/0054612 A1
(43) Pub. Date: Mar. 10, 2005

(54) Delivery by labile hydrophobic modification of drugs

(76) Inventors: Sean D. Monahan, Madison, WI (US); Vladimir G. Budker, Middleton, WI (US); Zane C. Neal, Cambria, WI (US); Vladimir Subbotin, Madison, WI (US)

Correspondence Address: MIRUS CORPORATION, 505 SOUTH ROSA RD, MADISON, WI, 53719, US

(21) Appl. No.: 10/929,697

(22) Filed: Aug. 30, 2004

Related U.S. Application Data
(60) Provisional application No. 60/501,189, filed on Sep. 8, 2003.
(60) Provisional application No. 60/520,426, filed on Nov. 14, 2003.
(60) Provisional application No. 60/513,707, filed on Oct. 23, 2003.
(60) Provisional application No. 60/558,753, filed on Apr. 1, 2004.

(51) Int. Cl. A61K 31/695; A61K 31/58; C07F 7/21; C07J 1/00

(52) U.S. Cl. 514/63; 514/176; 552/505; 556/407

(57) ABSTRACT

Described are drug formulations that increase regional delivery of the drugs to cells. Methods for reversibly increasing the hydrophobicity of a drug through hydrolytic ally labile attachment of a hydrophobic moiety and methods for delivering the modified drug to cells is described. Hydrophobic modification increases drug delivery, while lability minimizes entry of the drug into non-target cells.

### CROSS-REFERENCE TO RELATED APPLICATIONS

This application claims the benefit of U.S. Provisional Application No. 60/501,189, filed Sep. 8, 2003, 60/520,426, filed Nov. 14, 2003, 60/513,707, filed Oct. 23, 2003, and 60/558,753, filed Apr. 1, 2004.

### BACKGROUND OF THE INVENTION

A variety of methods and routes of administration have been developed to deliver pharmaceuticals to their site of action. The general problem associated with drug delivery is balancing ability to cross cell membrane with solubility in water. If a drug is too hydrophilic, it will be unable to cross the hydrophobic environment of the lipid cell membrane. If a drug is too lipophilic, it will be insufficiently soluble in an aqueous environment and possibly confined to the cell membrane if it does reach the cell. Most of the drug formulations are therefore amphiphilic, containing both hydrophilic and hydrophobic characteristics with a balance between charged and uncharged parts of molecule, or are formulated with the use of excipients to aid in the delivery of the drug.

Cellular drug delivery by conventional water-soluble drug formulations is limited by three obstacles regardless the route of administration: a) low partitioning through the cell lipid membrane, b) rapid clearance from a site of administration by the circulation, and c) redistribution throughout the body potentially leading to accumulation in unwanted tissue and systemic toxicity. Attempts to overcome the first two obstacles by increasing the dose of the drug increases systemic toxicity. To resolve these issues, the use of a regional delivery has been suggested. Such a delivery method is proposed to have the advantage of achieving a high drug concentration at the target site with a low systemic toxicity. The rationale of this approach is based on the pharmacokinetic principle of high first-pass extraction (Young 1999; Ensminger WD 1978). However, hydrophilic drugs, even delivered locally in high concentration, exhibit low partitioning through the lipid membrane and are cleared rapidly from the site of application (Morgan 2003).

Several types of diseases, notably several types of cancers, have been treated with a regional treatment regiment. For example, the regional treatment of liver cancers has been explored. The liver is the predominant site for metastatic disease progression from a variety of tumor origins, including colorectal carcinoma, melanoma, and neuroblastoma, and is the primary site for hepatocellular carcinoma (HCC) and cholangiocarcinoma (Alexander 2002). Systemic chemotherapy demonstrates poor antitumor benefit and only marginal increases in survival. Resection and transplantation remain the only curative options for patients with progressive liver disease (Iwatsuki 1999; Yamamoto 1999).

As liver neoplasms grow, tumors reaching a diameter of 5-7 mm are predominantly perfused by a neovascularized hepatic arterial route (Archer 1989; Laffer 1995). Normal liver parenchyma, however, is supplied mainly from the portal vein (75%). Exploitation of this difference motivated the development of regional treatment strategies such as direct hepatic artery infusion (HAI). However, analysis of a multicenter randomized trial indicated no differences in overall survival between HAI and systemic chemotherapy administration, and recommended discontinued HAI utility outside the scope clinical trials (Kerr 2003). Another regional therapy has been developed consisting of transcatheter hepatic artery chemotherapy (TAC) via the femoral artery (bolus injection), optionally combined with embolization (TACE). However, using conventional drugs, TACE has shown only modest patient benefit for both primary and secondary liver malignancies.

Additionally, a regional treatment for ovarian cancer has been investigated. Ovarian cancer is the second most common pelvic tumor and the leading cause of death from a gynecologic malignancy. Because of the lack of symptoms in the early stages, two thirds of patients present with advanced late-stage disease. Despite advances in surgical oncology, chemotherapy, and molecular biology, overall 5-year survival rates are still poor (approximately 30%).

Intraperitoneal chemotherapy (IPC) was introduced for peritoneal disseminated disease in an effort to direct high levels of chemotherapeutics to the peritoneal exposed tumor surface area. This treatment regime has been additionally modified as intraperitoneal perfusion chemotherapy (IPPC). IPPC removes unabsorbed drug from the peritoneal cavity to decrease systemic toxiciy and allow for higher dose administration of the chemotherapeutics. However, only modest benefits in disease remission and patient survival have been achieved for either IPC or IPPC.

Inefficient drug uptake combined with systemic and regional toxicity, and thus a narrow therapeutic index, results in the inability of these and other chemotherapies to effectively control the spread of microtumors and provide a survival benefit to patients.

The ability of chemotherapeutics to mediate cytotoxic activity is dependent on sufficient intracellular drug accumulation in the target cell. Intracellular drug levels are determined by passive membrane diffusion and active or facilitated import and efflux of the drug. It has been proposed that approximately one-half of all drug uptake, takes place by passive diffusion and the other half occurs by facilitated transport (Gately 1993).

The intracellular level of an antitumor drug, which directly influences the drug's cytotoxic effect, is a function of the amount of drug transported inside the cell (influx) and the amount of drug expelled from the cell (efflux). Drug uptake is determined by membrane transport, occurring through poorly defined mechanisms of passive diffusion and/or energy-dependent active transport. Passive diffusion and active drug transport are dependent on compatible interaction between cell membrane lipid-bilayer components and the drug molecule. Conventional drug formulations generally exhibit a hydrophilic character in order to resolve solubility issues (or have an excipient in the formulary, such as Cremphor) and demonstrate poor cellular uptake, even when delivered locally in high concentration, due to low partitioning through the lipid membrane. It has been thought that lipid membranes represent a barrier for hydrophilic drug movement, but are not a barrier for hydrophobic drugs.

Although liposome technology has facilitated better hydrophilic drug uptake, concerns regarding liposome accumulation in the reticulo-endothelial system and micelle aggregate formation questioned overall efficacy. An alternative approach is to modify the hydrophobicity of such hydrophilic drugs through chemical alterations of the parent drug. This idea of modification of parent drug has found a great deal of interest in recent years, with an ever greater number of modified drug delivery systems under development. As the intracellular accumulation of chemotherapeutics is critically dependent on the balance of influx and efflux, modifications in drug design that can impart sufficient lipophilicity and enhanced uptake should result in higher drug concentrations within the cell and positively affect antitumor responsiveness. Additionally, this increased drug concentration within the cell could help to alleviate drug resistance of tumor cells, as insufficient doses of drugs often initiates this drug resistance.

The attachment of hydrophobic groups, hydrophobation or lipidization, has been investigated in the area of drug and peptide/protein delivery (Wang et al. 2003; Tallen et al. 2000; Storch et al. 1996; Schreier et al. 2000; Kamyshny et al. 1997; Storch et al. 2002; Baszkin et al. 2001). These efforts have normally relied on the development of relatively stable or slowly cleaved modifications. Hydrophobation has been shown to increase interactions with cellular membranes (Wender et al. 2000; Yaroslavov et al. 1996; Gaede et al. 2003) and has been directly correlated with improved cellular uptake and lowered IC₅₀ determinations (McKeage et al. 2000; Gamier-Suillerot et al. 2001). Hydrophobic derivatives of cisplatin have been shown to have differences in cytotoxicity that parallel the hydrophobicity of the compound (Tallen 2000).

### SUMMARY OF THE INVENTION

Described are drug formulations that increase regional drug delivery to target cells. The drugs are modified via labile linkages to increase hydrophobicity, and thus membrane permeability. The resultant prodrug in stable in suitable solvent, but unstable in a suitable carrier solution. Just prior to administration of the prodrug to cells, the prodrug is mixed with a carrier solution. Lability of the prodrug in the delivery solution minimizes entry of the drug into non-target cells.

In a preferred embodiment, we describe the transient hydrophobic conversion of a drug into a prodrug for delivery to cells via first-pass delivery. Hydrophobic conversion increases membrane permeability of the prodrug. Lability of attachment of a hydrophobic moiety to the drug provides for limited duration of membrane permeability. Cleavage of the hydrophobic moiety after the association of the prodrug with the cell allows interaction of the unmodified drug with cellular components. Cleavage of the hydrophobic moiety outside the cell decreases the ability of the drug to enter cells and thus decreases undesired effects of the drug, such as toxicity, in non-target, i.e., non-first-pass, cells. A preferred hydrophobic moiety comprises a silazane. Another preferred hydrophobic moiety comprises a maleamic acid.

In a preferred embodiment we describe a method for delivering a hydrophobic drug or prodrug to a cell comprising: providing a prodrug that is soluble in an organic solvent, and injecting the prodrug in an organic solvent into a suitable mixing chamber designed to mix the organic solvent with a aqueous carrier solution just prior to delivery of a combined delivery solution to the cell. A suitable mixing chamber rapidly mixes the organic solvent with the aqueous carrier solution without producing laminar flow of the organic and aqueous solvents.

In a preferred embodiment, we describe compositions comprising: labile hydrophobically modified prodrugs that are soluble in organic solvents. Hydrophobic modification increases delivery of the drug to a cell interior. Lability results in rapid regeneration of the unmodified drug. The hydrophobic prodrug can be delivered to a cell by mixing the drug, in an organic solvent, with a sufficient amount of an aqueous carrier solution just prior to administration of the prodrug to the cell, cell container, or tissue. A preferred hydrophobic produg comprises a silazane modified drug. Another preferred hydrophobic produg comprises a maleamic acid.

In a preferred embodiment, we describe a method for increasing delivery of a drug to tumor cell comprising: hydrophobically modifying the drug via hydrolytically labile attachment of a hydrophobic group, mixing a solution containing the prodrug with a carrier solution by injecting the solutions though a mixing chamber just prior to delivery, and administering the combined solutions at or near the tumor cell.

In another preferred embodiment, we describe a method for the hydrophobic modification of a mixture of drugs (a drug library), via hydrolytically labile attachment of a hydrophobic group to the drugs, and the delivery of this prodrug library to cells. The hydrophobic prodrug library can be delivered to a cell by mixing the drug, in an organic solvent, with a sufficient amount of an aqueous carrier solution just prior to administration of the prodrug to the cell, cell container, or tissue.

In yet another preferred embodiment, we describe a method for the hydrophobic modification of a drug or mixture of drugs via the attachment of a labile hydrophobic group to the drug wherein the hydrophobic group is labile in response to a reaction by an agent, and delivery of this prodrug(s) to a cell. These hydrophobic prodrug(s) can be delivered to a cell by mixing the drug, in an organic solvent, with a sufficient amount of an aqueous carrier solution just prior to administration of the prodrug to the cell, cell container, or tissue. The agent can be a natural component of the cell or the environment of the cell or an agent added to the carrier solution.

Further objects, features, and advantages of the invention will be apparent from the following detailed description when taken in conjunction with the accompanying drawings.

### BRIEF DESCRIPTION OF THE DRAWINGS

FIG. 1. Illustration of hydrophobic modification of propidium iodide by chlorodimethyl-octadecylsilane (DMODSiCI) and 2-(dodecyl)propionamide-3-methylmaleic anhydride (CDMC 12).

FIG. 2. Illustrations of the chemical structures of Melphalan, BDMODS-Melphalan, the maleamic acid derivative CDMC12-Melphalan, Doxarubicin, DMODS-Dox, and the maleamic acid derivative CDMC12-Dox.

FIG. 3. mixing chamber (move to later figure, in examples; or include general delivery diagram as FIG. 1)

FIG. 4. Images of SK-OV-3 cells treated with: 1a&b—unmodified propidium iodide (PI); 2a&b—BDMODS-PI; 3a&b —CDMC12-PI; or 4a&b—pre-hydrolyzed BDMODS-PI. 1 a-4a: images under phase contrast illumination. 1b-4b: images of the same fields under fluorescent illumination with rhodamine filter.

FIG. 5. Images of Jurkat cells treated with (A) propidium iodide of (B) CDMC12-PI. Top panels show cells under phase contrast illumination. Bottom panels show the same field of cells under fluorescent illumination with rhodamine filter.

FIG. 6. Bar graph illustrating antiproliferative/cytotoxic effect of prodrugs on B 16 mammalian cells as measured by CellTiter-Glo luminescent cell viability assay.

FIG. 7. Confocal images illustrating propidium iodide delivery to cells in vivo following treatment with (A) BDMODS-PI and (B) CDMCl₂—PI. (A) Fallopian tube (B). Colon wall following. Propidium iodide (upper left panel of A and B), ToPro-3 nuclear stain (lower left panel of A and B); Actin stain with Phalloidin Alexa 488 (upper right panel of A and B).

FIG. 8. Light microscope images illustrating SK-OV-3 cancer cell growth 5 weeks after inoculation into nude mouse. (A) Micronodular growth on the duodenal mesentery, ×100. (B). Cancer cell formations on the pancreas and duodenum, ×200. (C) Cancer cells coating the liver surface, ×200. (D). Cancer cell coating and invading abdominal surface of diaphragm, ×200. Arrows indicate areas of tumor growth. Panels demonstrate the loose attachment of surface cancer cells to tumor mass. Hematoxylin-eosin stain.

FIG. 9. Confocal images of (A) BDMODS-PI and (B) CDMC12PI uptake by peritoneal ovarian tumors. (A) Small ovarian tumor growing on a visceral peritoneum close to the jejunum, ×630. (B) An ovarian tumor growing on and invading the colon surface, ×400. Propidium iodide (upper left panel of A and B), ToPro-3 nuclear stain (lower left panel of A and B); Actin stain with Phalloidin Alexa 488 (upper right panel of A and B).

FIG. 10. Confocal images following IPPC of CDMC12-PI: (A) Surface of a large peritoneal tumor, and (B) A micro-ovarian tumor on the surface of the colon, ×630; 5 weeks post SK− OV3 cell inoculation. Propidium iodide (upper left panel of A and B), ToPro-3 nuclear stain (lower left panel of A and B); Actin stain with Phalloidin Alexa 488 (upper right panel of A and B).

FIG. 11. Fluorescent images of liver sections following injection of modified propidium iodide (BDMODS-PI; A,B & D) or unmodified propidium iodide (C). A&B—Nuclei in MC38 metastases are strongly labeled with PI, as well as arteries and some adjunct cells following hepatic artery delivery. C—Few MC38 metastases labeled. D—No labeled cells in MC38 metastases following portal vein injection of BDMODS-PI. Images in the left column show propidium iodide fluorescence. Images in the right column show cell auto-fluorescence.

Arrowheads in C and D indicate border of tumor. HV=hepatic vein. A=100×; B, C, D=200×., Left panel—red channel (PI), right panel—green channel (autofluorescence).

FIG. 12. Delivery of BDMODS-PI to mouse liver with colon metastases. Left—×400 confocal image of liver with colon carcinoma tumors, arrow indicates portal tract with arteries labeled, arrowheads indicate liver metastasis with vast majority cells labeled. Right-×630 confocal image taken from the middle of metastasis, showing nearly all cells labeled with reported drug. Upper left panels—propidium iodide signal. Upper right panels—actin stained with Alexa 488. Lower left panels—ToPro-3 nuclear dye.

FIG. 13. First-pass delivery of labile hydrophobic drugs to: A. hepatic artery endothelial and smooth muscle cells; B. Gall bladder vascular and epithelial cells; C. bile duct epithelia and nearby hepatocytes; D. hepatocytes; E. endothelial cells and neurons; F. (control) liver following injection of unmodified propidium iodide into the hepatic artery; G. hepatic artery endothelia, smooth-muscle cells and tumors cells staining with modified propidium iodide; H. ureter transitional epithelia; I. renal pelvis transitional epithelia; J. beginning renal pelvis epithelia; K. collecting tubules; and, L. cornea epithelia.

### DETAILED DESCRIPTION

We describe drug formulations and processes for the delivering drugs into cells via a first-pass effect comprising: reversibly attaching one or more hydrophobic moieties to the drug via a very labile linkage to form a prodrug and bringing the modified drug into contact with the cells. The hydrophobic attachment imparts membrane permeability to the drug, thereby allowing the drug to enter a cell. The half-life of the hydrophobic attachment is comparable with the time necessary for first-pass delivery following single-bolus injection or the time necessary for drug diffusion after topical application. Thus, the prodrug is capable of permeating a cell membrane of a target cell for only a limited period of time. In one embodiment, the linkage attaching the hydrophobic group to the drug is stable in a compatible organic solvent but hydrolytically unstable in an aqueous environment. In another embodiment, the linkage attaching the hydrophobic group to the drug is more stable (longer halflife) in a basic environment but less stable as the pH is lowered. Because of the instability of the hydrophobic modification, drug that enters a cell as prodrug rapidly reverts to the original drug molecule which is then free to interact with target molecules. Prodrug that does not interact with cell membranes during first-pass rapidly reverts to the membrane impermeable drug through loss of the hydrophobic moiety. Reversion limits delivery of the drug into downstream cell thus limiting systemic toxicity.

The described drug modifications and processes can be used to enhance cellular accumulation of a chemotherapeutic drug in tumor tissue while decreasing systemic toxicity. The chemotherapeutic, or anti-neoplastic, is transiently converted into a lipophilic prodrug via labile chemical linkage of a hydrophobic moiety to the drug. Conversion of the drug to a prodrug promotes greater interaction with a cellular membrane. Rapid hydrolysis of the chemical linkage under physiological conditions restores the drug to the more membrane impermeable state associated with the parent drug. Transient lipophilic conversion facilitates enhanced drug uptake by tumor tissue and subsequent antitumor efficacy during first-pass delivery, while preserving low systemic toxicity by reversion to the parent drug prior to systemic exposure.

The hydrophobic modifications utilized in the prodrug formation are very labile, allowing for facile regeneration of the active drug within the cell. Because first-pass delivery serves to deliver more drug to regional target cells, such as tumor cells, lowering of the overall dosing of the drug may be possible. The rapidly labile prodrugs, which are highly cell permeable in a first-pass setting, are prevented from entering non-target cells further from a release site through rapid hydrolysis of the hydrophobic moiety. Prodrug that is not extracted during first-pass reverts to a relatively membrane impermeable drug. Thus, non-target cells are not exposed to the cell permeable prodrug. The result is a transient increase the therapeutic index of conventional chemotherapeutics while maintaining low systemic toxicity.

While hydrophobic modification of chemotherapy drugs to increase cellular interactions has been described in the art, we now show that the use of very hydrolytically labile hydrophobic modifications enable unique treatment scenarios with increased regional cell uptake of a modified drug following a single bolus injection while minimizing systemic exposure of non-target tissue to the active drug. These modifications are also useful for topical treatment of target cells while limiting drug exposure to non-target cells. We present an approach to the design of new drug formulations using reversible hydrophobic modifications and specialized delivery techniques that are capable of targeting compounds to desired tissues or organs while limiting interactions with non-target cells. The described chemistries and delivery methods also allow formation of prodrugs which are more hydrophobic, leading to better cell uptake and tumor penetration. A degree of hydrophobicity necessary to achieve cell delivery can be used without requiring that the produrg remain water soluble.

The lipophilic character of the prodrug, and thus its level of membrane interaction, will depend on the number and hydrophobicity of groups attached. Sufficient hydrophobicity is added to the drug to increase delivery of the resultant prodrug to cells. Hydrophobic groups indicate in qualitative terms that the chemical moiety is water-avoiding. Typically, such chemical groups are not water soluble, and tend not to hydrogen bond. Hydrocarbons are hydrophobic groups. If the hydrophobic group comprises an alkyl chain, the length of an alkyl chain group will affect the hydrophobicity of the group. Hydrohpobic groups compatible with the described invention may be selected from the group comprising: an alkyl chain of 4 to 30 carbon atoms, which may contain sites of unsaturation; an alkyl group containing an alkyl chain and alkyl rings (aromatic and/or non aromatic); and steroids. The linkages can also be designed such that they posses different lability rates in order to influence prodrug stability in vitro and in vivo. Limited stability of the drug modification allows for a local high concentration of modified drug that is able to enter cells in a first-pass region. A too rapid half-life results in ineffective target cell uptake. Conversely, a half-life of the prodrug that is too long leads to increased delivery of drug to non-target cells and tissues, potentially leading to systemic toxicity. The lability of the described linkages is potentially controllable through the choice of the pharmaceutically acceptable carrier solution. For example, the pH of the carrier solution can be adjusted with the use of an appropriate buffer in order to control the half-life of the prodrug. For drugs which can be modified with multiple hydrophobic groups, attachment of additional groups can not only increase the hydrophobicity of the drug, but also effectively increase the time required for complete hydrolysis. Controlling the incubation time of the drug between initial mixing with the carrier solution and initial contact with cells can also be used to influence the amount of time the lipophilic prodrug is present with cells. The rate of hydrolysis of the prodrug may be retarded upon interaction with the cellular membranes. The kinetic lability required for optimal delivery can be controlled through temperature or composition of the pharmaceutically acceptable carrier solution, the volume of the injection, the concentration of the injected prodrug, and the total amount of prodrug delivered.

We demonstrate the hydrophobic modification of amine-containing drugs via two different chemical linkages. An amine-containing drug has a nitrogen atom in the molecule that is amenable to modification. The amine can be a primary, secondary, or tertiary amine, or another nitrogen derivative such as an aniline.

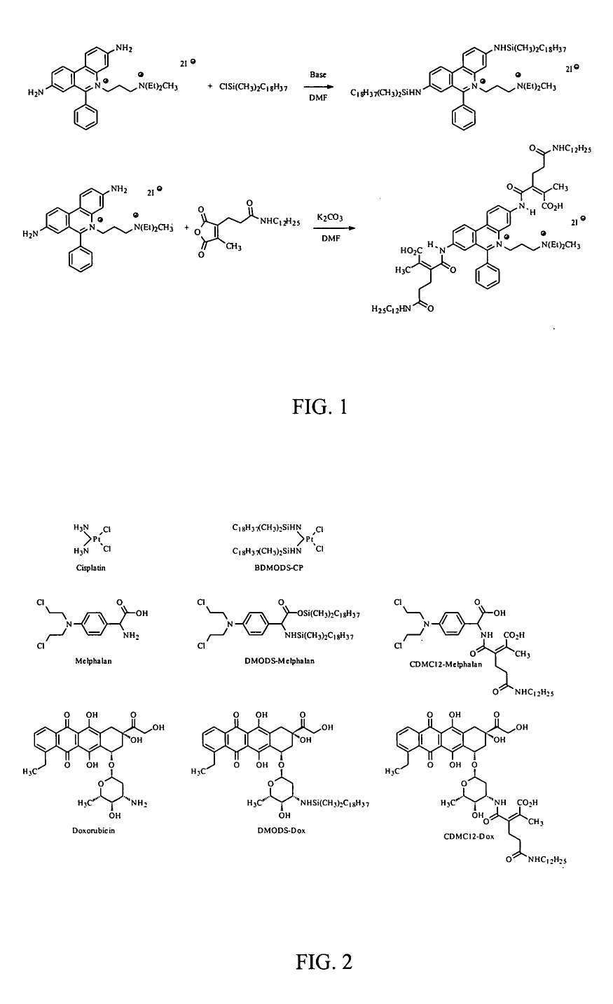

Amine containing drugs can be modified with silazanes. As an example, we show modification of drugs with chlorodimethyloctadecylsilane (DMODSiCI) to yield the corresponding dimethyloctadecylsilazane derivative as the hydrophobic prodrug (example shown in FIG. 1). The function of this group is to transiently attach hydrophobic groups to the drug molecule. The invention is meant to include other silazane derivatives. One skilled in the art will readily recongize that a variety of silazanes can be employed to impart transient hydrophobicity (for example, including but not limited to: trimethylsilyl and tert-butyl-dimethylsilyl groups).

The reaction between an amine and a chlorosilane is a well-known chemical modification which forms a silazane (or silylamine). Silazanes are known to hydrolyze rapidly in the presence of water to yield the original amine and a silanol or disilyl ether (Jiang et al. 2002; Jiang et al. 2002b; Lucke et al. 1997; March 1992; Greene et al. 1999; Kulpinski et al. 1992; Prout et al. 1994). Silazanes have generally been utilized in the field of ceramics or in organic synthesis as reagents for the silylation of other functional groups, most notably, the hydroxyl group. Because of its lability, this modification has not found utility in biological applications. However, more stable heterosilanes have been employed as prodrugs. Examples include: a trimethylsilyl ether of testosterone; silabolin, a per-trimethylsilylated derivative of dopamine; carbosilane drugs; and silicon used as part of a delivery system (Brook 2000; Brahim et al. 2003; Nouvel et al. 2003; Nouvel et al. 2002; Bom et al. 2001; Perkins et al. 1994; Kratz et al. 1999). These examples employ a stable bond (carbon-silicon) or a slowly hydrolyzed bond (silicon-oxygen), not a rapidly hydrolyzed bond as found in the silazane. Silyl ethers have long been utilized as removable protecting groups in organic synthesis. The bond is hydrolytically labile under acidic conditions to yield an alcohol and a silanol or disilyl ether. Several factors control the hydrolysis rate of silyl ethers, for example the sterics of the silicon atom (ie the bulk of groups attached to silicon), and the pH of the solution. Silyzanes (with the exception of the known stable variants) hydrolyze much more readily than the corresponding oxygen variants (the silyl ethers).

For some drugs, direct silylation will prove inefficient. In such cases, other approaches can be used to form the prodrug (Ohya, 2001; Reedijk, 1999; Tallen, 2000; Alvarez-Valdes, 2002). For example, in the case of cisplatin, DMODSiCl can be reacted with methylamine to form dimethyloctadecylsilyl-methylamine. This silazane can then be added to cisplatin or Pt(DMSO)₂-1,1-cyclobutanedicarboxylate to yield a labile cisplatin derivative. Silylation of a heterocyclic nitrogen atom is also possible.

Amine containing drugs can also be modified with maleic anhydrides possessing hydrophobic groups. As an example, we show modification of drugs with 2-(dodecyl)-propionamide-3-methylmaleic anhydride to yield the corresponding hydrophobic prodrugs. (example shown in FIG. 2) Maleic anhydrides have been previously utilized for reversible amine modification (Naganawa, 1994; Reddy, 2000; Dinand, 2002; Hermanson, 1996). The resulting maleamic acids are known to be stable under basic conditions, but hydrolyze rapidly under acidic conditions. For example, 2-propionic-3-methylmaleic anhydride (a carboxylic acid derivative) has been tested with glycinylalanine. The resulting maleamic acid has been shown to have a half-life of 2 min at pH 5 (k=0.3 min⁻¹) (Rozema, 2003). Given that aniline nitrogen's are generally less reactive than amines due to delocalization with the aromatic ring, it was expected that the lability of an aniline derived maleamic acid would be greater than that of the maleamic acid derived from a primary amine. As with the silazane, the purpose of the maleic anhydride is to transiently attach a hydrophobic groups to a drug molecule. One skilled in the art readily recognize that a variety of maleic anhydrides can be employed to impart transient hydrophobicity.

A great number of labile bonds are known to those skilled in the art, that could be utilized to attach a hydrophobic group or moiety to a drug molecule. The invention is also meant to encompass the use of hydrophobic drug modifications with these other types of hydrolytically labile bonds, when the derived prodrugs are then delivered via the delivery methods described in the present invention. Examples of additional labile bonds that may be used to attach the hydrophobic moiety to the drug include, but are not limited to: imines, ortho esters, acetals, aminals, sily esters, and phosposilyl esters. The pH of the carrier solution can be adjusted in order to effect the halflife of the prodrug formulation. Preferably, the invention encompasses hydrophobically modified drug formulations in which the halflife of the modification is less than or equal to 5 minute in the delivery solution. Hydrophobic drug modifications with shorter halflives in the delivery solution, less than 1 minute, less than 30 seconds, or less than 20 seconds, may be preferred.

The methods described herein are also compatible with perfusion technology. In this context, perfusion refers to the deliberate introduction of fluid into a tissue. The fluid can be introduced into a vessel, tissue lumen, body cavity, such as the peritoneal cavity or in vitro cell container. More specifically, in isolated perfusion, the perfused tissue is isolated such that the introduced fluid does not reach nontarget tissues. The isolated tissue can be flushed both before and after the perfusion to remove bodily fluid or introduced fluid from the tissue or region. Perfusion has been used to deliver anti-cancer agents into the blood vessels and tissues of an organ (liver or lung) or region of the body (usually an arm or a leg) using circulating bypass machines. Such a procedure is performed to treat cancer that has spread but is limited to an organ or region of the body. In the context of the present invention, the prodrug (dissolved in drug carrier solvent) is mixed with an aqueous carrier solution in a mixing chamber and delivered to a the tissue to be perfused. An outflow line permits the prodrug delivery solution to perfuse through the cavity and exit through the outflow line. Because the prodrug and drug (resulting from loss of the hydrophobic group(s)) are removed from the tissue, it is possible to utilize prodrugs with a longer halflives than in cases where the material is not removed following delivery. When the prodrug—drug is not removed, it is preferred to have a prodrug with a shorter halflife in order to protect downstream cells from the highly cell permeable prodrug. In the case of isolated perfusion, the prodrug is removed from the area of interest, thereby protecting cells outside the target region.

The described produgs are synthesized in organic or other appropriate solvents. The described prodrugs are also stable in these solvents but hydrolytically unstable in a carrier or delivery solution, such as an aqueous solution. The reaction to form the modified drug can be conducted in a variety of solvents, however, an injectable solvent is preferred. Furthermore, a solvent in which the modified drug can be purified from other components of the modification reaction (for example, hydrolyzed hydrobobic group, drying agents, and bases) is preferable to facilitate purification of the prodrug.

A variety of drugs can be modified according to the invention. Preferably, the drug would be modified through an amine group on the drug. These drugs may be selected from the list comprising: chemotherapeutics, anti-neoplastic, doxorubicine (adriamycin), cisplatin (cis-diamminedichloroplatinum(II)), melphalan, the tubulin polymerization agent paclitaxel. Additional functional groups that can be modified include alcohols, thiols, phosphates, and carboxylates. An active derivative of the parent drug, which contains a functional group suitable for modification may also be used. Examples of modified drugs include: cisplatin derivatives containing a heterocyclic nitrogen, anthracycline derivatives of doxorubicin, and amino or furanosyl substituted 5-fluorouracil.

We further describe methods for delivering labile produgs comprising: co-injecting the prodrug in an organic or other suitable solvent (a drug carrier solvent) together with an aqueous carrier solution though a mixing chamber. Many of modified drugs describe herein have hydrophobic groups attached by very hydrolytically reactive linkages that require synthesis and storage in organic solvents. However, toxicity concerns prohibit the direct delivery of drugs to cells in undiluted organic solvents. Therefore, mixing the organic solvents with a pharmaceutically acceptable aqueous carrier solution just prior to delivery by co-injection though a mixing chamber is performed.

The critical components of a suitable mixing chamber include: means by which to accurately deliver predetermined volumes of drug carrier solvent and aqueous carrier solution, means to rapidly and intimately mix the drug carrier solvent and aqueous carrier solution, and a means of delivering the combined liquid (delivery solution) to cells. Some commercial mixing chambers can result in laminar flows, without effective mixing of the drug carrier solvent with the carrier solution. If the drug carrier solvent is an organic solvent, incomplete mixing results in exposure of some cells to too high a concentration of organic solvents leading to membrane damage. If the mixing is too slow, then the prodrug may be cleaved prior to contact with the cells. Any mixing chamber that provides adequate and rapid mixing of the drug carrier solvent with the aqueous carrier solution is suitable for use with the present invention.

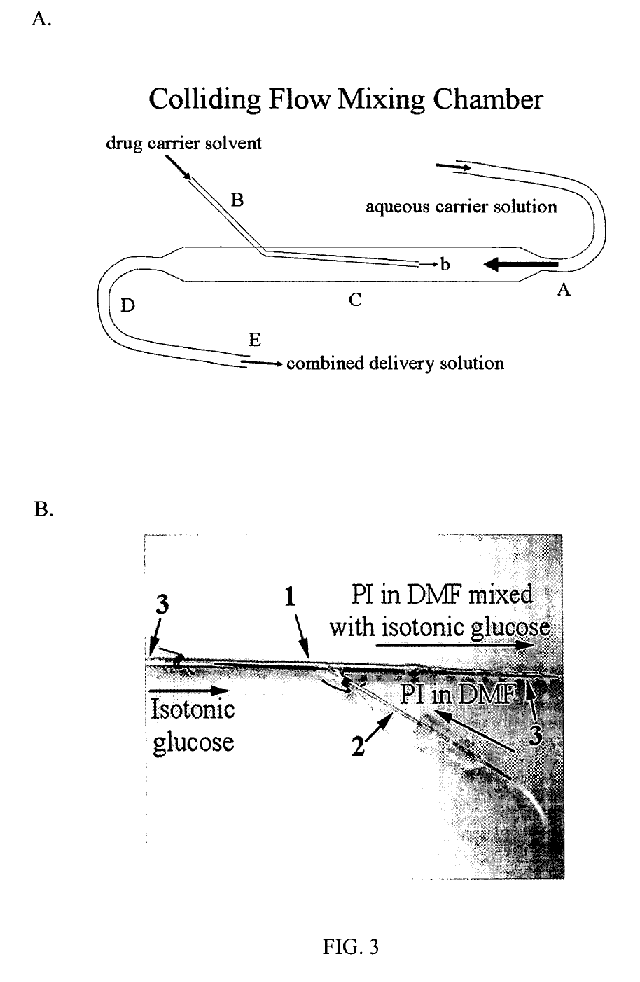

An example of a suitable mixing chamber is the colliding flow mixing microchamber shown in FIG. 3. The aqueous carrier solution and the drug-carrier solvent are injected into a mixing chamber (C) though conduits (A) and (B) respectively. The direction of flow (b) of the drug carrier solvent into chamber (C) is in the opposite direction of the flow of the aqueous carrier solution into chamber (C), facilitating mixing of the two liquids. The combined delivery solution is then delivered to cells through vessel conduit (D) and instillation port (E). The volume of drug carrier solvent is generally much less than the volume of carrier solution. In one version of the mixing chamber, a Harvard Pump PHD 2000 with a 100 μl Hamilton syringe and a Harvard Pump PHD 2000 with a 1 ml Becton Dickinson syringe were used to accurately deliver small volumes to the chamber. Conduits (A), (B), and (D) may be rigid or flexible and may be made of any material than is suitable to convey the respective solutions and drugs. The length of conduit (D) may be varied in length to alter the amount of time the prodrug is in the aqueous carrier solution prior to delivery to the cells, thus modulating the halflife of the prodrug in the presence of the cells. A longer deliver route increases the incubation time and therefore decreases the halflife of the lipophilic modified drug in contact with the animal and/or on the cells. Suitable instillation ports (E) may be selected from the list comprising syringe needles and catheters.

Mixtures detailed in following examples contain 1/10^{th} volume prodrug in organic solvent mixed with 1 volume aqueous carrier solution (such as, but not limited to, Ringer's or isotonic glucose (ITG)). The total volume of prodrug-containing solvent to be delivered should be less than that which would cause toxicity from the solvent. The volume of carrier solution should be chosen to provide adequate total volume for the target area and provide adequate dilution of the prodrug-containing solvent. For larger animals, target areas, or cell containers, increased volume is appropriate. The volume and rate of injection of the combined delivery solution should be less than that which would cause significant damage to the cells from the pressure alone.

The membrane permeability and lability of the prodrug (i.e. the halflife of the modified drug) can be measured by monitoring the uptake of the prodrug by liposomes. The eleunt from a suitable mixing chamber can be delivered to a solution containing liposomes whose composition approximates the plasma membrane of the target cells. The liposomes are then purified and the level of drug in the liposomes is measured. For DNA intercalating drugs, the liposomes can contain DNA to facilitate determination of drug uptake.

The described processes and prodrugs are readily compatible with known techniques such as regional hepatic artery infusion (HAI) therapy, intraperitoneal chemotherapy (IPC), intraperitoneal perfusion chemotherapy (IPPC), transcatheter hepatic artery chemotherapy (TAC), transcatheter hepatic artery chemotherapy with embolization (TACE), and isolated organ or tissue perfusion. In isolated perfusion applications, potential systemic toxicity of the drug is further reduced because unabsorbed hydrolyzed drug is flushed from the isolated tissue (such as a peritoneal cavity) prior to restoration of normal fluid flow through the tissue. The describe processes and prodrugs are also compatible with topical delivery of the drug.

While the process may be described as a single bolus delivery of the prodrug, the process is not limited to a single administration. The process may be repeated to provide for increased levels of drug delivery. The term single bolus delivery is meant to be descriptive of first-pass delivery of the drug following an injection/application of a predetermined quantity of the prodrug.

The describe prodrugs and methods can be used to generate an antitumor response against a variety of tumors, both primary and secondary, including, but not limited to, hepatocellular carcinoma, colon carcinoma, melanoma, ovarian carcinoma, and neuroblastoma. The utility of single bolus delivery is dependent on the ability of the drug agent to be preferentially exposed to the neoplastic tissue and penetrate the tumor cell membrane during first-pass delivery. Modification of anticancer drugs through labile attachment of hydrophilic moieties transforms relatively membrane impermeable drugs into lipophilic prodrugs that facilitate increased intracellular drug concentrations and enhanced anticancer responses.

For some cancers, such as peritoneal cancer, cancer cells and microtumors are invariably present together with the detectable and operable metastases. Their presence and continuous defoliation from primary and secondary malignancies represent one of the main impediments to the successful treatment of cancers such as peritoneal disseminated ovarian cancer. The disclosed prodrug formulations target all exposed cells, single cells, microinfiltrates, microtumors, and surface cells of larger peritoneal tumors, tumor cells suspended in peritoneal cavity or attached to or invading an organ or tissue. The described prodrugs also exhibit increased penetration of the drug into tumors compared to conventional drugs. We have observed drug penetration up to 500 μm (about 25 cell layers) within seconds. Thus, the described formulations provide for improved delivery of anticancer drugs to cancer cells in a variety of states. The described invention could therefore be utilized following cytoreductive surgery in efforts to slow or minimize reappearance of tumors.

Various types of tissues can be targeted using the described invention, depending on the type of delivery method utilized. For example, hepatocytes are targeted with a single bolus injection to the portal vein (occluded blood flow). Following a single bolus injection into the hepatic artery of normal mouse liver, targeting was evident in the hepatic artery endothelial and smooth-muscle cells, and in a few neighboring hepatocytes and sinusoidal cells. All biliary and gall bladder arteries, as well as bladder epithelium also were targeted. Bile duct cells together with some hepatocytes are targeted following a single bolus injection to the bile duct. Urinary tract cells are targeted (ureter transitional epithelium nuclei, renal pelvis transitional epithelium nuclei, including beginning renal pelvis, that is the source for transitional cell carcinoma, and a majority of collecting tubules, and other epithelial compartnents) following a single bolus injection to the ureter. A single bolus injection into the carotid artery of a normal mouse resulted in the targeting of brain endothelial cells and both neurons and glial cells. Topical administration of prodrug results in delivery to the cells to which the produge is directly applied. For example, topical application to the cornea or to a skin or into the lumen of the intestine results in drug delivery to the cornea epithelium, or epidermis, or enterocytes respectively.

To further increase delivery of drugs to cells, the described formulations and process may be combined with co-delivery of compounds known to modulate drug efflux pump efficiency. This co-delivery serves to increase drug retention in the cell.

The present invention is also applicable to the modification and delivery to cells of mixtures of drugs, also know as drug libraries. Most drugs contain nitrogen or oxygen atoms within the molecule that aid in the solubility of the drug in aqueous solutions. These atoms can be hydrophobized according to the procedures outlined in this specification. The drug library can be taken up in an appropriate organic solvent such as DMF or DMSO, and be subjected to hydrophobic modification such as outlined for a single compound. The derived prodrug library can then be applied to cells as outlined for a single prodrug.

The present invention is also applicable to a method for the hydrophobic modification of a drug or mixture of drugs via the attachment of a labile hydrophobic group to the drug wherein the hydrophobic group is labile in response to a reaction of an agent. For example the hydrophobic group can contain a disulfide bond which, upon entry to the cell, will be cleavable by the cellular agent glutathione. Hydrohpobic groups compatible with the described invention contain a disulfide bond at or within 4 carbon atoms of the point of attachment of the group to the drug molecule that is susceptible to reduction by glutathione. The disulfide system also possesses a hydrophobic group on one side of the disulfide bond that may be selected from the group comprising: an alkyl chain of 4 to 30 carbon atoms, and can contain sites of unsaturation, an alkyl group contining an alkyl chain and alkyl rings (aromatic and/or non aromatic), and steroids. The linkages can also be designed such that they posses different lability rates in order to influence prodrug stability in vitro and in vivo.

The term drug in the present invention is also meant to include the pharmaceutically acceptable salt of the drug. Pharmaceutically acceptable salt means both acid and base addition salts. A pharmaceutically acceptable acid addition salt is a salt that retains the biological effectiveness and properties of the free base, is not biologically or otherwise undesirable, and is formed with inorganic acids such as hydrochloric acid, hydrobromic acid, sulfuric acid, nitric acid, phosphoric acid and the like, and organic acids such as acetic acid, propionic acid, pyruvic acid, maleic acid, malonic acid, succinic acid, fumaric acid, tartaric acid, citric acid, benzoic acid, mandelic acid, methanesulfonic acid, ethanesulfonic acid, p-toluenesulfonic acid, salicylic acid, trifluoroacetic acid, and the like. A pharmaceutically acceptable base addition salt is a salts that retains the biological effectiveness and properties of the free acid, is not biologically or otherwise undesirable, and is prepared from the addition of an inorganic organic base to the free acid. Salts derived from inorganic bases include, but are not limited to, sodium, potassium, calcium, lithium, ammonium, magnesium, zinc, and aluminum salts and the like. Salts derived from organic bases include, but are not limited to, salts of primary secondary, and tertiary amines, such as methylamine, triethylamine, and the like.

A labile bond is a covalent bond that is capable of being selectively broken. That is, the labile bond may be broken in the presence of other covalent bonds without the breakage of the other covalent bonds. For example, a disulfide bond is capable of being broken in the presence of thiols without cleavage of other bonds, such as carbon-carbon, carbon-oxygen, carbon-sulfur, carbon-nitrogen bonds, which may also be present in the molecule. Labile also means cleavable.

A labile linkage is a chemical compound that contains a labile bond and provides a link or spacer between two other groups. The groups that are linked may be chosen from compounds such as biologically active compounds, membrane active compounds, compounds that inhibit membrane activity, functional reactive groups, monomers, and cell targeting signals. The spacer group may contain chemical moieties chosen from a group that includes alkanes, alkenes, esters, ethers, glycerol, amide, saccharides, polysaccharides, and heteroatoms such as oxygen, sulfur, or nitrogen. The spacer may be electronically neutral, may bear a positive or negative charge, or may bear both positive and negative charges with an overall charge of neutral, positive or negative.

### EXAMPLES

### Example 1

Labile hydrophobic modifications of propidium iodide. Propidium iodide was utilized as a model reporter-drug. This membrane impermeable reporter drug is routinely used as a fluorescent agent to visually identify cells possessing compromised membranes. Cell with intact cellular membranes effectively exclude propidium iodide. Propidium iodide exhibits a 20-30-fold enhanced fluorescence upon intercalation into DNA, facilitating detection of propidium iodide positive (PI⁺) cells. Stable attachment of a hydrophobic group to propidium iodide (by alkylation of the aniline with dodecyl bromide) prevents propidium iodide intercalation and staining. The ability to deliver a fluorescent test drug to tumors provides a valuable visual tool in evaluating many experimental parameters.

Modifications of propidium iodide with chlorodimethyloctadecylsilane (DMODSiCI) and 2-(dodecyl)propionamide-3-methylmaleic anhydride (CDMC12) to form bis-(dimethyloctadecylsilyl)-propidium iodide (BDMODS-PI) and the bis maleamic acid derivative (CDMCl₂—PI); and, modification of cisplatin with DMODSiCl to form bis-(dimethyloctadecylsilyl)-cisplatin (BDMODS-CP) (FIG. 2).

A. Synthesis of BDMODS-PI. Hydrophobic modification of propidium iodide (95%, Aldrich Chemical Company) was accomplished by treating propidium iodide with an excess of DMODSiCl (6 molar equivalents, Aldrich) in anhydrous N,N-dimethylformamide (DMF, Aldrich) or dimethyl sulfoxide (DMSO, Aldrich), with K₂CO₃ as a base, and activated 3A molecular sieves present as a water scavenger, to form BDMODS-PI (FIG. 2A). Prior to use, the BDMODS-PI is filtered through a 0.20 μm sterile Nylon filter, which removes the solids and the hydrolysis products of the DMODSiCl (which are not DMF soluble). Although DMF and other amides are known to react with chlorosilanes, no evidence of the imidate (or products arising from the imidate) have been observed under these reaction conditions (79-82). In order to verify the structural assignments, several additional controls (silylations and alkylations) were also conducted on related aniline ring systems (including aniline and 3,8-diamino-6-phenylphenanthridine). All products were analyzed by 1H NMR (250 MHz, N,N-dimethylformamide-d7), and support the structural assignments. The NMR indicated the addition of two alkylsilane groups to the PI based on integration and a corresponding loss of two aniline protons. The effective rate of hydrolysis of the BDMODS-PI also was too rapid to be tested by NMR.

Kinetic Analysis—The related anilinium salt of BDMODS-PI was analyzed for kinetic measurements. The addition of H₂O to the DMF solution of the anilinium salt resulted in a rapid decrease and shift in the maximum fluorescence from 645 nm to 655 nm (Ex 500 nm). The t_{1/2} for the reaction can be measured as approximately 43 sec, affording a rate constant for the reaction of k=0.016 sec^{-l}, based upon the equation t_{1/2}=ln2/k. From these data, an approximate halflife of the silazane in pure water can be estimated to be 0.4 sec. Although the hydrolysis is very rapid, it is expected that this is a faster hydrolysis than what would be expected for BDMODS-PI due to the positive charge on the aniline nitrogen, leading to our estimate of a slightly slower hydrolysis rate for BDMODS-PI. It is possible that trace dimethylamine (present in DMF) causes rapid hydrolysis of the BDMODS-PI upon dilution of the sample for analysis, resulting in little apparent difference between BDMODS-PI and PI. Testing with DMSO as the solvent allowed for the direct monitoring of the hydrolytic breakdown of BDMODS-PI. The addition of buffer (25 μL, 50 μL, and 100 μL of 20 mM Hepes, pH 8.5 and 7.2) to BDMODS-PI in DMSO (Ex 493 nm, Em 647 nm) resulted in a loss of the silyl groups and an appearance of fluorescence at 647 nm. Based upon the t_{1/2} for the reactions, a rate constant for the reaction of kₐₚₚₐᵣₑₙₜ=0.103 sec⁻¹ (Hepes buffer at pH 8.5) can be approximated, and a halflife of the BDMODS-PI in pure buffer can be estimated to be 6.7 sec according to the equation t_{1/2}=ln2/k. The hydrolytic lability of BDMODS-PI was much faster as the pH of the solution was lowered to pH 7.2. Based upon the t_{1/2} for the reactions with Hepes buffer at pH 7.2, a rate constant for the reaction of kₐₚₚₐᵣₑₙₜ=0.478 sec⁻¹ can be approximated, and a halflife of the BDMODS-PI in pure buffer can be estimated to be 1.5 sec. The rapid hydrolysis of modified compound back to parent drug agent is important for increasing the therapeutic index of these alkylating chemotherapeutics in the context of first-pass delivery to tumors.

Micelle or Particle Formation Upon Mixing of the Solutions. It is plausible that BDMODS-PI forms a micellar type particle when the drug/DMF solution is mixed with the ITG carrier solution. In order to investigate this possibility, particle sizing experiments were conducted (ZetaPlus Particle Sizer, Brookhaven Instrument Corporation), which indicated that the present modification chemistries did not promote particle formation following chamber mixing with ITG.

B. Synthesis of CDMC12-PI. Propidium iodide was also modified with the cyclic anhydride CDMC 12 to form the bis-maleamic acid derivative CDMC 12-PI (FIG. 2A). The reaction of a maleic anhydride and an amine is a reversible reaction, favoring the acylation (the maleamic acid) under basic conditions. Upon acidification, maleamic acids are know to undergo ring closure to yield the original amine and the original anhydride. This ring closure reaction of maleamic acids is generally slow under neutral conditions. However, in the case of aniline-like nitrogens, as found in propidium iodide, the hydrolytic lability of the bond is predicted to be much more facile under neutral conditions than what would be found with a normal amine.

Aniline nitrogens are generally less reactive (and have lower pKa's) than other amines due to delocalization of electron density with the aromatic ring.

In the current modification reaction with propidium iodide (not optimized), CDMC12 (3 eq) was added together with K₂CO₃ in DMF or DMSO at 60° C. Analysis of the reaction by 1H NMR (250 MHz, N,N-dimethylformamide-d7) indicates the loss of the amine protons of propidium iodide and the appearance of the amide protons from the bis-maleamic acid. Control reactions have been conducted with both aniline and 3,8-diamino-6-phenylphenanthridine in order to verify structural assignments.

Analysis of the hydrolytic breakdown of the CDMC12-PI was accomplished by fluorescence spectroscopy (Ex 493 nm, Em 647 nm) in DMSO. From these data, a rate constant could be approximated for the reaction of kₐₚₚₐᵣₑₙₜ=0.086 sec⁻¹ (Hepes buffer at pH 8.5), and a lifetime halflife of the CDMC 12-PI in pure buffer can be estimated to be 8.1 sec. Even at basic pH, the halflife of the maleamic acid is short, due to the low pKa of the aniline type nitrogen on the PI. As expected, the hydrolytic lability of CDMC12-PI increased as the pH of the solution was lowered to pH 7.2. Based upon the t_{1/2} for the reactions with Hepes buffer at pH 7.2, a rate constant was approximated for the reaction of kₐₚₚₐᵣₑₙₜ=0.113 sec⁻¹, and a halflife of the CDMC 12-PI in pure buffer is estimated to be about 6.1 sec.

### Example 2

### Labile Hydrophobic Modification of Chemotherapeutics

A. Labile hydrophobic modifications of cisplatin, synthesis of BDMODS-CP. Cisplatin was utilized as a model conventional chemotherapeutic. A similar series of silylation reactions were carried out on cisplatin (cis-diamminedichloro-platinum(II); Fuertes, 2003; Reedijk, 1999; Siddik, 2003), a widely used platinum-based chemotherapeutic, to yield Cl₂Pt(NH₂Si(CH₃)₂C₁₈H₃₇)₂ (BDMODS-CP). More stable hydrophobic modifications have previously been investigated with cisplatin, usually by replacement of the amine portion of the molecule with a substituted amine, in order to modify activity and toxicity (Tallen et al. 2000; Alvarez-Valdes et al. 2002). Analysis of the reaction (not optimized) of cisplatin and DMODSiCl by ¹H NMR (250 MHz, N,N-dimethylformamide-d7) indicated the loss of the broad amine signal at δ4.2 (relative to TMS) and the appearance of the alkyl groups from the reaction with the chlorosilane. Integration of the relative signals indicated 1.7 alkyl groups per cisplatin molecule. This material was filtered through a 0.20 μm sterile Nylon filter.

B. Synthesis of DMODS-Melphalan and CDMC12-Melphalan. A similar series of modifications were carried out on the chemotherapeutic melphalan, utilizing both DMODSiCl and CDMC12 to form BDMODS-Melphalan and the maleamic acid derivative CDMC 12-Melphalan (FIG. 3). The reactions (not optimized) were carried out in either DMF or DMSO utilizing a slight excess of modification reagent in the presence of K₂CO₃ and activated 3 Å molecular sieves as a water scavenger. Given the differences in functional groups present in melphalan, different labile modifications are possible. For example, melphalan has a tertiary amine, a primary amine, and a carboxylic acid. Given the reactivity of DMODSiCI, two modifications are expected to occur, one on the primary amine, and the second with the carboxylic acid to form a silylester. The acylation reaction with 2-(dodecyl)propionamide-3-methylmaleic anhydride, to form the maleamic acid will only occur on the primary nitrogen of melphalan. Analysis of the reaction by ¹H NMR (250 MHz, N,N-dimethylformamide-d7) supported modification of the melphalan as described.

C. Synthesis of DMODS-Dox and CDMC12-Dox. Preliminary (not optimized) modifications of doxorubicin, utilizing both DMODSiCl and CDMC12, have been initiated to form DMODS-Dox and the maleamic acid derivative CDMC 12-Dox (FIG. 3). Doxorubicin hydrochloride (98%, Aldrich) was reacted with 1-4 equivalents of DMODSiCI or CDMC 12 in DMF in the presence of base. The modified drug was qualitatively examined by liposomal uptake and cellular toxicity assay, which indicated that as the number of equivalents of modification agent increased, more of the doxorubicin was sequestered into a liposome and those liposomes imparted greater cytotoxicity when applied to Hepa 1-6 cells in vitro. Control experiments also indicated the prodrug formulations themselves (non-liposomal) demonstrated enhanced cytotoxicity in the cell experiments compared to doxorubicin (data not shown).

### Example 3

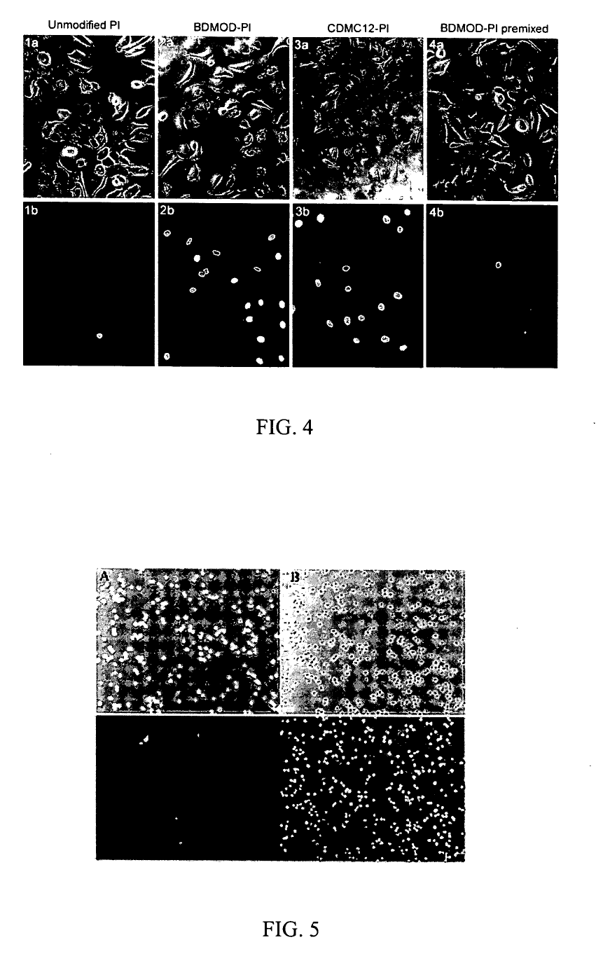

BDMODS-PI and CDMC12-PI stain viable targets. We tested the ability of BDMODS-PI and CDMC12-PI to stain viable cells (both tumor cells and lymphocytes), indicating successful propidium iodide-prodrug uptake, intracellular release of functional propidium iodide, and DNA intercalation. SK-OV-3 (human ovarian carcinoma, FIG. 4), Jurkat (human T-lymphocyte; FIG. 5; plated on polylysine coated cover slips in the well), and Hepa 1-6, MC38 (mouse hepatoma and colon carcinoma, data not shown) cells were plated at 0.25×10⁶ cells/well in 6-well plates 24 hrs prior to media removal and addition of the drug or prodrug. Unmodified propidium iodide (FIG. 4, panel 1A & 1B), BDMODS-PI (FIG. 4, panel 2A & 2B), CDMC12-PI (FIG. 4, panel 3A & B), and BDMODS-PI premixed for 5 min with ITG (FIG. 4, panel 4A & 4B) were added to the SK-OV-3 cells. All drug concentrations were constant at 100 μg propidium iodide in 20 μL DMF/200 μL isotonic glucose carrier solution and delivered using the described missing chamber. After 30 seconds the drug solution was aspirated and 2 mL of fresh media was added. Each condition was repeated in 3 wells.

Cells were immediately examined with an Axiovert S100 fluorescent microscope. Unmodified propidium iodide stained very few cells (FIG. 4, panel 1), representing normally occurring dead cells in the population. BDMODS-PI and CDMC 12-PI stained 60-80% of the cells (FIG. 4, panels 2-3), demonstrating that the hydrophobically modified prodrugs efficiently enter viable human ovarian cancer cells with successful intracellular formation of active free propidium iodide. Premixing of BDMODS-PI in ITG, which permits hydrolysis of the labile linkage and release of propidium iodide did not show PI⁺staining (FIG. 4, panel 4B). Similar results were also obtained from the uptake experiments with Hepa 1-6 cells. In the case of Jurkat cells (human T-lymphocyte), propidium iodide and CDMC 12-PI were added to the wells in duplicate. After 3 minutes the drug solution was diluted by adding 2 mL of fresh media. Unmodified propidium iodide stained very few cells, while CDMC 12-PI exhibited strong cellular uptake and staining (essentially 100% of the cells, FIG. 5). Similar experiments carried out on a number of cell lines including MC38 (mouse colon carcinoma), B16 (mouse melanoma), SK-OV3 (human ovarian carcinoma), and HeLa (human cervical carcinoma), resulted in 60-80% of the cells exhibiting PI uptake (using BDMODS-PI).

### Example 4

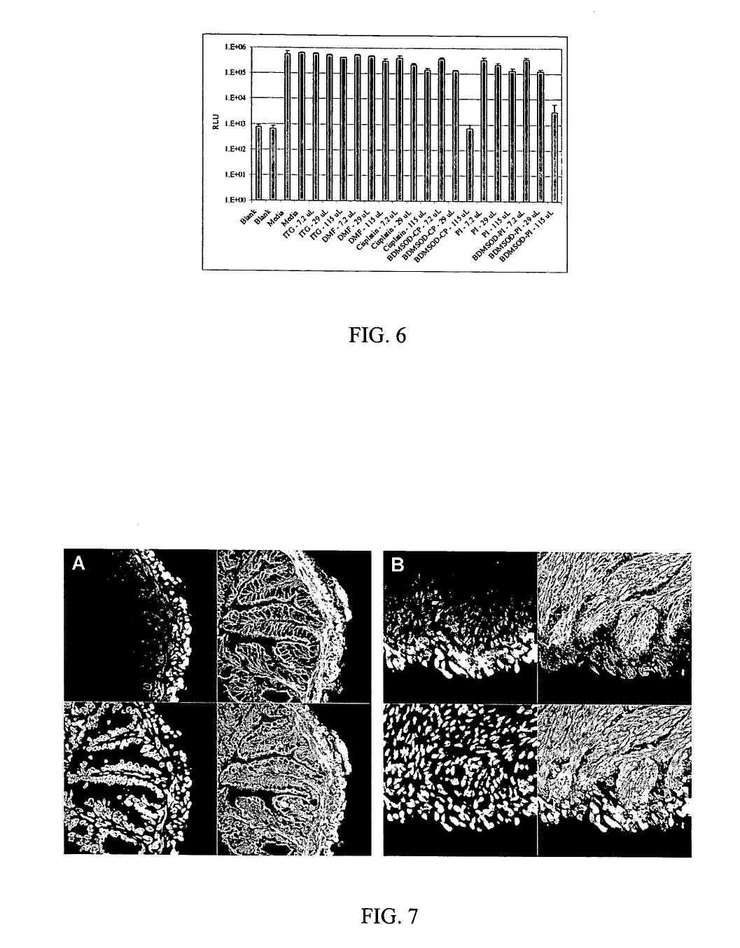

Enhanced antiproliferative/cytotoxic effect of hydrophobic-modified cisplatin and melphalan. To determine whether hydrophobically modified drugs demonstrate enhanced antitumor activity, we performed in vitro cytotoxicity testing on B 16 murine melanoma cells and MC38 colon carcinoma cells. Using the dual pump colliding flow mixing chamber delivery system for drug delivery, we evaluated the effect of propidium iodide, BDMODS-PI, cisplatin, and BDMODS-CP on B 16 cells using the CellTiter-Glo luminescent cell viability assay (FIG. 6). Cells were seeded at 1×10⁴ cells/well in 100 μl of media into 96-well plates on day 0 and cultured for 24 hr prior to addition of drug. Following removal of media, drug solution was added drop-wise (1, 4, or 16 drops; with effective delivery of 7, 24, and 112 μl, respectively, of solution) to quadruplicate wells, using a 1:11 DMF/ITG solution ratio with the dual pump mixing chamber. Following 10 min of drug solution exposure, 100 μl of fresh media was added to each well and incubated for 3 hrs. Drug-containing media was replaced with 100 μl of fresh media and cultured for and additional 24 hr. Data represent the mean RLU values of quadruplicate wells+S.D. Drug concentrations evaluated: cisplatin (2.5 μg/μl DMF), propidium iodide (5.0 μg/μl DMF).

The ITG carrier solution, as well as DMF, exhibited negligible antiproliferative effects against B 16 cells when compared to media only controls. Cisplatin showed a mild dose-dependent antiproliferative response, with maximal effect at the highest drug level tested (24 μg drug in 112 μL of DMF/ITG delivery solution per well). In comparison, the modified cisplatin prodrug markedly enhanced the antiproliferative and/or cytotoxic activity of the drug. At the highest level tested, BDMODS-CP reduced RLU levels to those observed with blank wells (media only wells without B 16 cells), indicating complete cytotoxic effect against the B 16 tumor cells.

CDMC 12-Melphalan also demonstrated enhanced antiproliferative/cytotoxic activity against MC38 murine colon carcinoma cells. As shown in FIG. 6, DMF exhibited negligible effect on the growth of the MC38 cells as compared to media only control. Melphalan induced a dose dependent growth inhibition, with doses of 1.9 and 5.8 μg having similar antiproliferative effects. At the lower 0.6 and 1.9 μg doses, CDMCI2-Melphalan exhibited growth inhibition similar to melphalan. At the 5.8 μg dose, CDMC12-Melphalan exhibited significantly (P=0.00086) greater growth inhibition when compared to the same dose of melphalan (mean RLU values of 187400 and 685543, respectively).

These results clearly indicate that hydrophobic modifications of cisplatin and melphalan facilitates enhanced antitumor effects against melanoma and colon carcinoma cells in vitro.

### Example 5

BDMODS-PI and CDMC12-PI show enhanced drug uptake by surface tissue following IP application. All in vivo procedures were executed under Isoflurane inhalation anesthesia in semi-sterile conditions. For evaluation of prodrug delivery to exposed tissues in the peritoneum, we tested IP application of propidium iodide, BDMSODS-PI, and CDMC 12-PI to both normal mice and in a mouse model of disseminated peritoneal ovarian cancer. All procedures were executed under Isoflurane inhalation anesthesia. In normal mice either the abdominal cavity was opened and the drug mixture was directly applied on abdominal organs (220 μL of drug-OS/ITG over 30 seconds), or the drug mixture was injected through the abdominal wall (1 mL of drug-OS/ITG over 1 min). For both delivery protocols, the dual pump mixing chamber was used. After 10-60 min, the animals were euthanized and tissues were harvested, frozen, sectioned, stained with ToPro-3 and Phalloidin Alexa 488, and examined by laser confocal microscopy. Application of propidium iodide in normal mice resulted in extremely rare nuclear labeling (data not shown), while application of both BDMODS-PI, and CDMC 12-PI resulted in near-exclusive PI⁺-staining of cells exposed to the peritoneal cavity. The cells situated deeper in the tissues appeared to be labeled at a much lower intensity or not at all (FIG. 7).

### Example 6

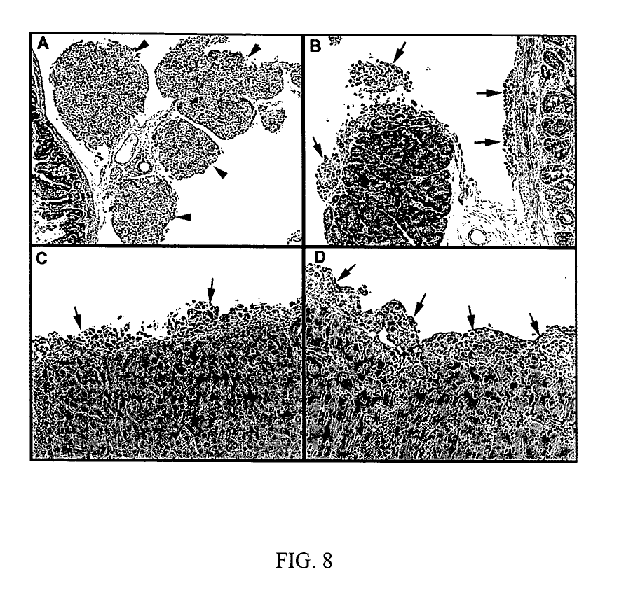

BDMODS-PI and CDMC12-PI show enhanced drug uptake by surface tissue and microtumors following IP and IPPC application in a mouse tumor model. For establishment of the cancer model, 2×10⁶ SK-OV-3 cells were injected IP into nude mice. The mice were examined at two weeks following cell inoculation, or at the first manifestation of ascites (about 4-5 wks). Tissue samples were fixed in 10% NBF, routinely processed, stained with H&E stain, and subjected to histopathological analysis. Microscopic examination indicated that at two weeks after SK-OV-3 cell inoculation, multiple microtumors (about 1 mm) were present throughout the peritoneal cavity, most notable on the mesentery. At 4-5 weeks after SK-OV-3 cell inoculation, maximum tumor size increased to 5-7 mm with the bulk of cancer development present as about 0.1 to about 1 mm microtumors (FIG. 8). At 5 wks most peritoneal surfaces were affected by growing cancer cells, coating both the visceral and parietal peritoneum, e.g. liver, pancreas, and diaphragm. Thus, the histopathological analysis indicated strong similarities in peritoneal perpetuation and dissemination between the SK− OV-3 mouse model and clinical ovarian cancer.

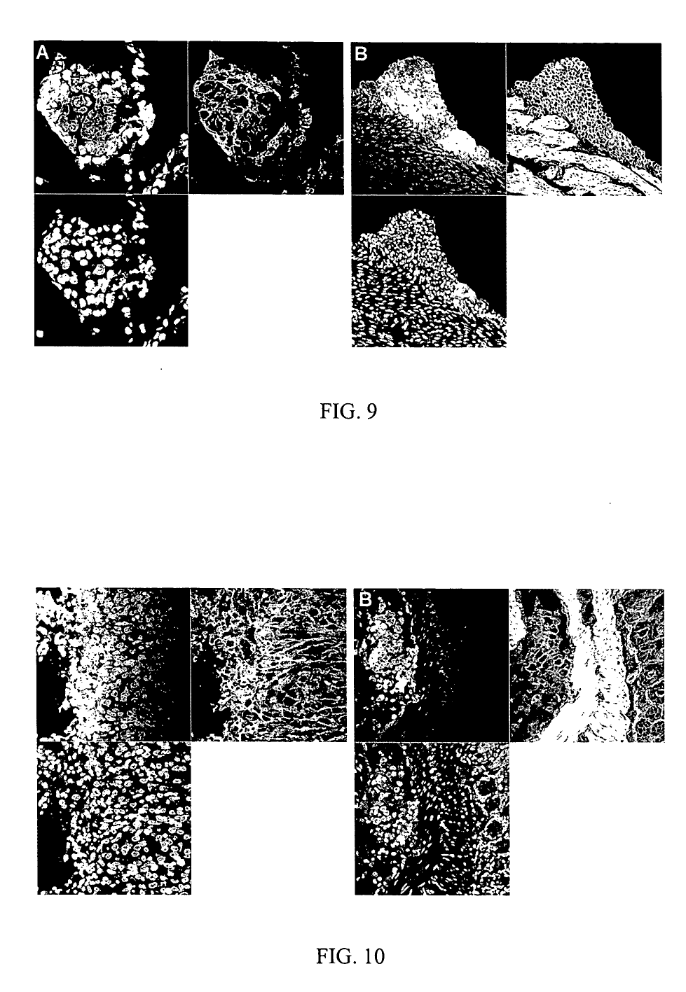

Two weeks following cell inoculation, the abdominal cavity was opened and the drug mixture was directly applied on the duodenum and ovary/uterus/fallopian tubes (220 μL of drug-OS/ITG over 30 seconds). Alternatively, the drug mixture was injected through the abdominal wall (1 mL of drug-OS/ITG over 30 seconds) as previously described. Both delivery routes utilized the described mixing chamber. After 10-60 min, animals were euthanized, tissue sections were harvested, and frozen sections were stained with ToPro-3 and Phalloidin Alexa 488, and examined by laser confocal microscopy. Direct application and the IP injection of both BDMODS-PI and CDMC12-PI resulted in similar observations. Cellular uptake of the prodrug was stronger at the surface of the metastases, but also readily detectable in the middle of the small tumors (tumor size about 0.5 to about 1 mm; FIG. 9). In addition to labeling relatively large tumors, smaller tumors (about 25-100 cells in cross-section), growing on the visceral peritoneum and mesentery were also intensely labeled with BDMODS-PI and CDMC12-PI. Adjacent mesentery cells indicated prodrug uptake and staining, as did the outer layer of cells of most of the normal tissues exposed to the peritoneal cavity and the prodrug solution (e.g., liver). However, the tumor lesions appeared to be more susceptible to prodrug uptake, showing greater tissue penetration and intense PI⁺-staining as compared to non-malignant tissues. Extensive sectioning and analysis indicated that tumors of any size were effectively targeted (up to 500 μm from the tumor surface). Tumors without propidium iodide-staining were not observed. In normal mice controls, similar analyses indicated near-exclusive PI⁺-staining of the outer cells exposed to the peritoneal cavity. Cells situated deeper in the tissues were labeled at a much lower intensity or not at all (FIG. 7).

In a separate experiment, ovarian cancer development was monitored until signs of ascitis (4-5 wks). At this time, a test IPPC injection was conducted with CDMC12-PI by adapting methodology typically used for peritoneal perfusion. Briefly, two 23 G Abbocath-T effluent catheters with multiple perforations were inserted into the peritoneal cavity and advanced to the region of the ovaries on both sides of the vertebra. The ascitic fluid was slowly aspirated with minimal negative pressure. Then an additional 23 G perforated catheter was inserted into abdomen and positioned on top of abdominal organs. 1 mL of drug/DMF/ITG solution was infused over 1 min, followed by repeated gentle massage. 5-7 min post administration, peritoneal fluid was again aspirated, and the peritoneal cavity was perfused with 10 mL of PBS via the top catheter, together with simultaneous aspiration via the lower two catheters. Special care was taken to avoid elevated abdominal pressure during the procedure. All animals survived the perfusion well and were sacrificed 3-5 hrs later. Confocal microscopy indicated a similar staining pattern as observed previously, with strong propidium iodide nuclear labeling of all of the outer cells exposed to peritoneal cavity, including ovarian tumors. Large tumors (5-7 mm) were labeled to a depth of about 500 microns (FIG. 10A), while all tumor cells in microtumors (0.1-1 mm) were heavily labeled throughout the tumor (FIG. 10B). All cells exposed to peritoneal cavity and thereby to the prodrug solution indicated prodrug uptake and staining, including: mesentery cells, outer layer of cells of abdominal organs, and disseminated peritoneal ovarian tumors. The tumor lesions still appeared to be more susceptible to drug uptake, indicating both greater tissue penetration and intense PI⁺-staining as compared to normal abdominal tissue, with the exception of the mesentery which was also was heavily PI⁺.

Generally, tissue penetration of small molecular weight drugs is difficult to accomplish. Several studies have indicated tissue penetration depths (many tumor types) for a variety of antineoplastics on the order of hours to days for 50-500 μm penetration. In contrast, our prodrugs resulted in a 500 μm penetration depth within ten minutes. This could be a result a more effective interaction of the hydrophobic prodrug with the cell membrane, similar to what is described in the literature as lateral diffusion.

Although rapidly hydrolysable prodrug was used for this experiment, more slowly hydrolysable prodrugs can be used with isolated perfusion delivery methods without increasing systemic toxicity.

### Example 7

Enhanced prodrug uptake by hepatic metastases following single bolus injection. In order to avoid hepatic artery mechanical damage and post-procedure contraction, we used the right gastro-duodenal artery for retrograde single bolus infusion to the hepatic artery in a manner similar to the clinical procedure (Kemeny, 2001). The gastro-duodenal artery was freed from surrounding tissue and the common hepatic artery and/or celiac was clamped occluding blood flow. The distal part of the gastroduodenal artery was sutured and a 35 G needle was inserted into the gastroduodenal artery and secured during the single bolus injection. Following injection, the needle was retracted, the proximal part of the gastroduodenal artery was sutured, and hepatic artery flow was restored. Alternatively, the celiac trunk was clamped close to the aorta and a 35 G needle was inserted above the clamp. The left gastric, splenic, and gastro-duodenal arteries were clamped in order to direct all of the drug solution to the liver. This latter approach was advantageous in the C57BL mouse model because of anatomical variations involving the hepatic artery.

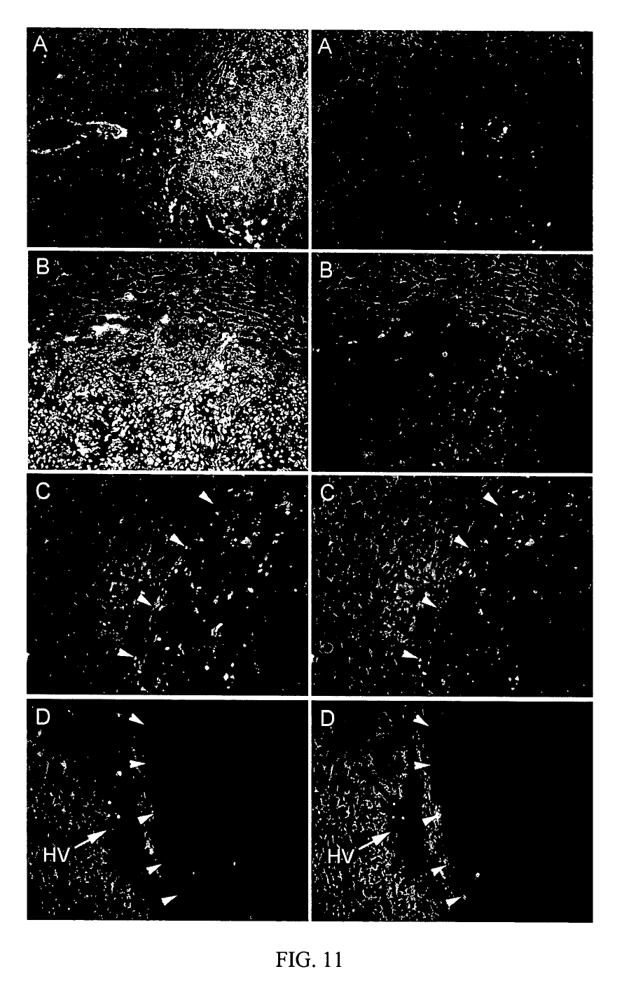

For evaluation of drug delivery to liver tumors, the murine colon cancer cell line MC38 was used. C57BL mice were inoculated with 104 MC38 cells via the portal vein and tumor formation was allowed to develop for three weeks. Prodrug or controls were delivered by bolus injection using the described mixing chamber. 100 μg BDMODS-PI in DMF at 5 mg propidium iodide per ml DMF was combined with ITG at a rate of 0.67 μl DMF per 6.7 μl ITG per second to deliver a total volume of 220 μl over 30 sec. Livers were harvested 5 minutes following drug delivery. As shown in FIG. 11, A & B, left panel, delivery of BDMODS-PI resulted in intense, near-exclusive PI-staining of MC38 liver metastases, while normal parenchyma appeared relatively free of PI staining.

Hepatic arteries and some adjunct cells were also labeled, as were a few cells at the parenchyma-metastasis interface. No residual PI⁺ cells were observed in any organ downstream from the liver in any of the BDMODS-PI treated animals. As expected, when unmodified PI or pre-hydrolyzed BDMODS-P was delivered by bolus injection, it resulted in very few cells (about 1%) stained with PI either in MC38 metastases or in parenchyma distal or proximal to tumor tissue (FIG. 11C, left panel). As an additional control, the same amount of modified PI was injected into the portal vein with preserved portal flow to the tumor-bearing mice. All metastases were PI-negative, with a few hepatic cells indicating uptake (FIG. 11D, left panel). When intraportal delivery was again performed with the portal vein clamped, temporarily occluding blood flow, prominent labeling of portal structures and adjunct cells, but not metastases, was noted. FIG. 11A,B,C&D, right panels, represent autofluorescence of the same fields in green channel.

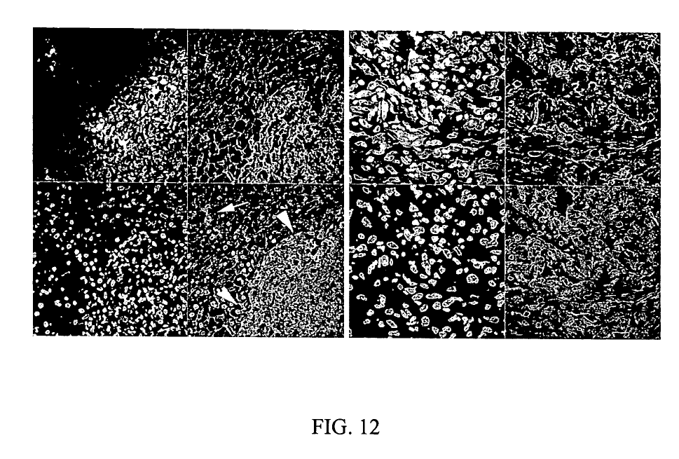

Confocal images of samples following LABC therapy were used to approximate the numbers of PI⁺ cells in three different compartments: in MC38 metastases, in the portal vein areas of the liver, and in the liver parenchyma with sinusoid vasculature (FIG. 12). Five images (0.262 mm² each) were obtained for each compartment from a lobe with MC38 tumors and the total number of nuclei (ToPro-3-positive staining) and PI⁺ nuclei were determined. The average percent of PI⁺ cells in MC38 metastases was 94%. Analysis also indicated that 23% of the cells in portal areas were PI⁺. These cells were mostly arterial cells together with some adjoining hepatocytes and bile duct cells (all of which were potentially exposed to BDMODS-PI during the injection). Given that portal areas comprise less than 5-7% of the liver, the actual number of PI⁺ cells is very small compared to positive cells in the metastases. Finally, analysis indicated the overall about 1% of the cells in the liver parenchyma were PI⁺. From these data a total estimation of the number of positive non tumor cells in the liver would be 2-3%.

We also evaluated intraportal delivery of BDMODS-PI in MC38 tumor-bearing animals. When portal blood flow was preserved during injection, it resulted in infrequent single-cell labeling of metastatic and normal parenchyma tissue. However, when intraportal delivery was performed with the portal vein clamped, temporarily occluding blood flow, prominent labeling of portal structures and adjunct cells, but not metastasis, was noted.

In addition to the MC38 colonic carcinoma model, we also performed preliminary testing of BDMODS-PI delivery in other relevant syngeneic murine liver metastases models, including Hepal-6 (hepatoma), B16 (melanoma), and NXS2 (neuroblastoma). Similar patterns of PI-staining were observed, with preferential staining of hepatic metastatic tissue and very minimal involvement of parenchyma, clearly indicating that the utility of this novel drug-delivery system is not restricted to a particular type metastatic liver disease. Additionally, we did not observe any residual PI⁺ cells, either in the lung or in heart of the BDMODS-PI treated animals (data not shown).

### Example 8

Uptake of BDMODS-PI by hepatocytes in vivo. In order to quantitate the amount of PI that enters cells in vivo, we intraportally delivered unmodified PI or BDMODS-PI to the liver in order to target hepatocytes. Under anesthesia both the portal and hepatic artery flows were clamped and the blood in the liver was flushed out with 1 ml of ITG via the portal vein. Then 200 μg of PI or BDMODS-PI in 4011 of DMF were mixed (via microchamber) with 400 μl of ITG and delivered intraportally. Five minutes after injection the livers were perfused with 3 ml of ITG to flush any drug remaining in the vasculature, and hepatocytes were isolated. Smears from cell suspensions were prepared and examined by fluorescent microscopy. Analysis of the smears indicated that PI labeled about 1% of the cells, while BDMODS-PI labeled about 70% of the cells. Cell suspensions were dissolved in 0.5% solution of octyl glycoside in 10 mM HEPES pH 7.5.

PI fluorescence spectra were monitored on a Shimadzu RF 1501 Spectrofluorimeter using an excitation wavelength of 530 nm. The amount of PI in each sample was estimated according to PI calibration curves generated by mixing increasing amounts of PI in each sample. Linear standard curves were obtained, indicating that PI binding to DNA was not saturated, thus allowing for accurate determination of the amount of PI present in the samples. The efficiency of PI uptake was then calculated according to the equation: Ef(%)=100×PIₛ/PIᵢ, where PIₛ is the amount of PI in the cell suspension and PIᵢ is the amount of PI or BDMODS-PI injected (corrected for the sample size relative to the total liver). These results indicated that 1.2% of the injected normal PI is bound to liver DNA versus 13.5% for BDMODS-PI.

### Example 9

Propidium iodide delivery to a variety of target cells. Propidium iodide was hydrophobically modified as described above and delivered via injection or topical administration using the mixing chamber and as detailed below.

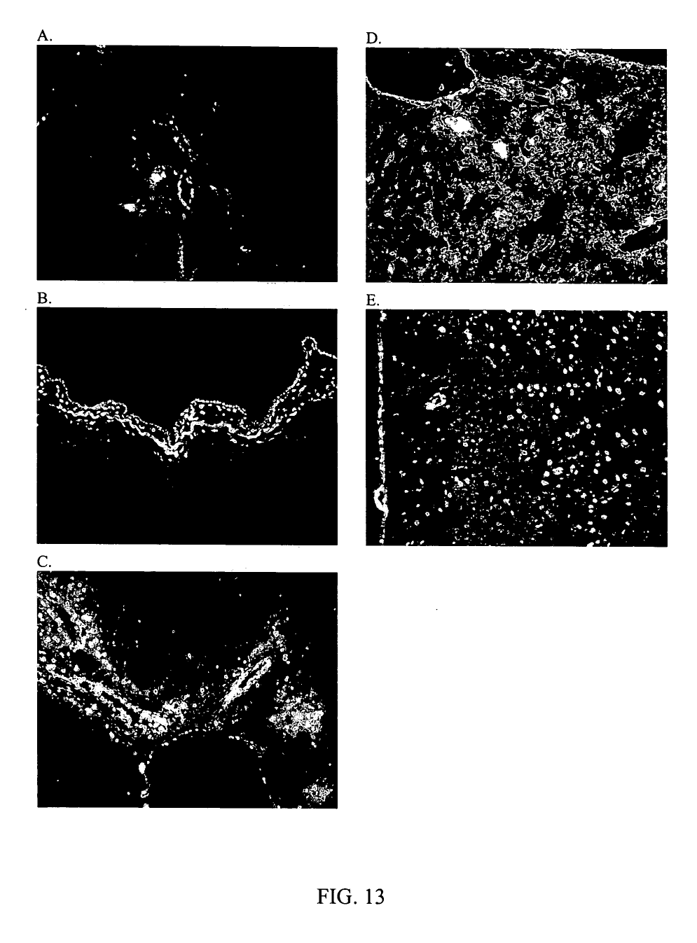

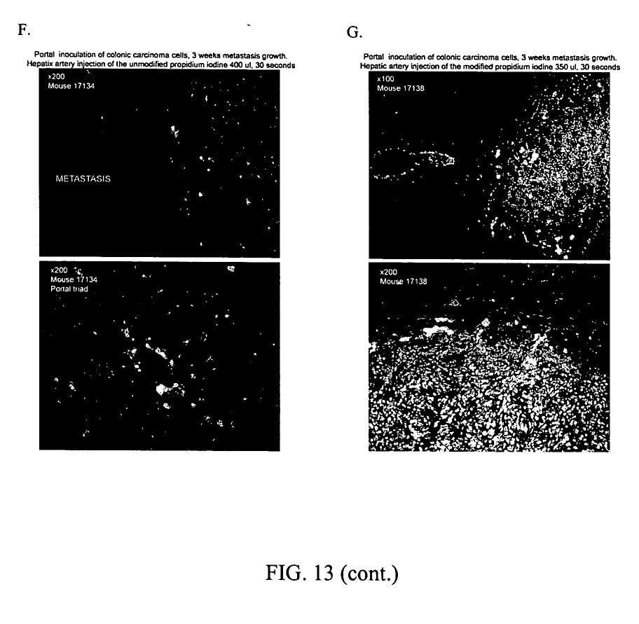

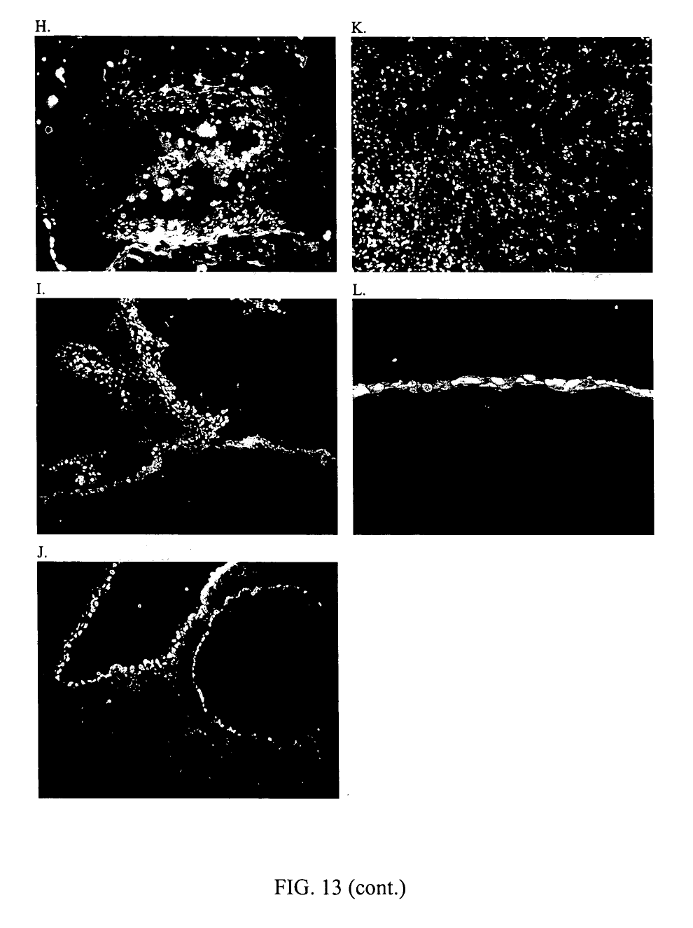

A. Injection into the hepatic artery of normal mouse liver: Injection of unmodified propidium iodide into any vessel or bile duct of normal liver resulted in little to no nuclear staining. Injection of modified propidium iodide into the hepatic artery of normal mouse liver resulted in strong nuclear staining of hepatic artery endothelial and smooth-muscle cells (FIG. 13A), and stained a few neighboring hepatocytes and sinusoidal cells. All biliary and gall bladder arteries, as well as bladder epithelium also stained (FIG. 13B). The technique described above was utilized.

B. Injection into the bile duct of normal mouse liver: Injection of modified propidium iodide into bile duct of normal mouse liver resulted in strong nuclear staining of all bile duct epithelial cells as well as staining in hepatocytes near the bile duct (FIG. 13C).

C. Injection into the portal vein of normal mouse liver: Injection of modified propidium iodide into unclamped liver portal vein with preserved blood flow of normal mouse resulted in strong nuclear staining of portal vein cells only. When the portal vein was clamped during the injection, prodium iodide staining was observed in a majority of hepatocytes (FIG. 13D).

E. Injection into the right carotid artery of normal mouse: 35 G needle was inserted into right common carotid artery then advanced into internal carotid. During injection time the common carotid artery was temporary occluded. After injection the blood flow was restored and animals sacrificed 5 minutes later. Injection of modified propidium iodide into right carotid artery of normal mouse resulted in strong staining of brain endothelial cells and in strong staining of both neuron and glial nuclei (FIG. 13E).

D. Injection into the hepatic artery of mouse liver with cancer metastases: Injection of unmodified propidium iodide into the hepatic artery of mouse liver with cancer metastases did not result in nuclear staining of any structures (FIG. 13F). However, injection of modified propidium iodide into hepatic artery of mouse liver with cancer metastases resulted in strong nuclear staining of hepatic artery endothelial and smooth-muscle cells, and in strong nuclear staining of the metastases (FIG. 13G).

F. Injection into the ureter and bladder of normal mice: Injection of modified propidium iodide into the ureter of normal mice resulted in strong staining of ureter transitional epithelium nuclei (FIG. 13H), renal pelvis transitional epithelium nuclei (FIG. 13I), including beginning renal pelvis (FIG. 13J), and a majority of collecting tubules (FIG. 13K). Injection was performed using similar 35 G needle into right ureter close to the bladder. Injection into emptied bladder resulted strong staining of bladder transitional epithelium.

G. Application on the right cornea of normal mice: Using the described above technique, the topical application to the cornea of normal mice resulted in strong nuclear staining of cornea epithelium only (FIG. 13L).

H. Application on skin surface of normal mice: Using the described above technique, the topical application to a skin of normal mice resulted in strong nuclear staining of epidermis predominantly (data not shown).

I. Intestinal intra lumen application of the BDMODS-PI. About 25 mm of duodenum and jejunum were clamped proximally and distally then 100 μg of PI or BDMODS-PI in 20 μl of DMF were mixed with 200 μl ITG and injected into lumen over 30 s, using the technique described above. Clamps were then released and 5 min later animals were sacrificed. The intestine lumen was flushed with 1 ml of ITG and analyzed as described above. While PI staining was observed in a few intestinal villi cells (presumably apoptotic cells), application of BDMODS-PI resulted in strong labeling of all enterocyte nuclei and significant portion of mesenchymal cells near enterocytes (data not shown).

The foregoing is considered as illustrative only of the principles of the invention. Furthermore, since numerous modifications and changes will readily occur to those skilled in the art, it is not desired to limit the invention to the exact construction and operation shown and described. Therefore, all suitable modifications and equivalents fall within the scope of the invention.

1. A modified drug comprising: a drug covalently linked to one or more hydrophobic moieties via one or more labile bonds.

2. The modified drug of claim 1 wherein said hydrophobic group is selected from the list consisting of: an alkyl chain of 4 to 30 carbon atoms, an alkyl group containing an alkyl chain and alkyl rings, and steroid.

3. The modified drug of claim 2 wherein said modified drug in soluble in organic solvent.

4. The modified drug of claim 3 wherein said labile bond consists of a hydrolytically labile bond.

5. The modified drug of claim 4 wherein said hydrolytically labile bond is selected from the list consisting of: a silazane or a maleamic acid.

6. The modified drug of claim 1 wherein said modified drug is cell membrane permeable.

7. A method for delivering a drug to a cell comprising:
a) attaching a hydrophobic group to said drug via a labile bond to form a prodrug,
b) dissolving said prodrug in a prodrug carrier solvent;
b) mixing said solvent with an aqueous carrier solution to from a delivery solution; and,
d) contacting said cell with said delivery solution.

8. The method of claim 7 wherein said labile bond consists of a hydrolytically labile bond.

9. The method of claim 8 wherein said hydrolytically labile bond is selected from the list consisting of: a silazane and a maleamic acid.

10. The method of claim 7 wherein said labile bond consists of a bond that is cleaved by a component of said aqueous carrier solution.

11. The method of claim 7 wherein said labile bond consists of a bond that is cleaved by a component of said cell.

12. The method of claim 7 wherein said hydrophobic group is selected from the list consisting of: an alkyl chain of 4 to 30 carbon atoms, an alkyl group containing an alkyl chain and alkyl rings and steroid.

13. The method of claim 12 wherein said prodrug in soluble in organic solvent.

14. The method of claim 7 wherein said prodrug is cell membrane permeable.

15. A method for delivering a hydrophobic compound to a cell comprising:
a) taking up said hydrophobic compound in an organic solvent;
b) mixing said organic solvent with an aqueous carrier solution to form a delivery solution; and,
c) adminstering said delivery solution to said cell wherein said hydrophobic compound associates with said cell.

16. The method of claim 15 wherein said hydrophobic compound comprises a molecule linked to a hydrophobic group via a labile bond.

17. The method of claim 16 wherein said labile bond consists of a hydrolytically labile bond.

18. The method of claim 17 wherein said hydrolytically labile bond is selected from the group consisting of: a silazane or a maleamic acid.

19. The method of claim 16 wherein said labile bond is cleaved by a component of said aqueous carrier solution.

20. The method of claim 16 wherein said labile bond is cleaved by a component of said cell.

21. The method of claim 16 wherein said hydrophobic group is selected from the list consisting of: an alkyl chain of 4 to 30 carbon atoms, an alkyl group containing an alkyl chain and alkyl rings and steroid.
